(19) United States
(12) Patent Application Publication
(10) Pub. No.: US 2025/0378962 A1
(43) Pub. Date: Dec. 11, 2025

(54) INFORMATION PROCESSING SYSTEM AND INFORMATION PROCESSING METHOD

(71) Applicant: I'mbesideyou Inc., Tokyo (JP)

(72) Inventors: Shozo KAMIYA, Tokyo (JP); Kotaro ANDO, Tokyo (JP)

(21) Appl. No.: 19/212,677

(22) Filed: May 20, 2025

(30) Foreign Application Priority Data
Jun. 10, 2024 (WO) PCT/JP2024/20991

(51) Int. Cl. G16H 50/30; G06T 7/00

(52) U.S. Cl. CPC G16H 50/30; G06T 7/0012; G06T 2207/10016; G06T 2207/20081; G06T 2207/30004

(57) ABSTRACT

An information processing system capable of estimating a user's mental state. The system comprises: a detection unit that analyzes a video image capturing a user and detects a degree of an attribute of the user; a calculation unit that calculates a second variation degree of a first variation degree related to the degree of the attribute in the video image; and an estimation unit that estimates a mental state of the user based at least on the second variation degree.

### TECHNICAL FIELD

The present invention relates to an information processing system and an information processing method.

### BACKGROUND OF THE INVENTION

A technology for analyzing emotions received by others in response to a speaker's remarks is known (for example, see Patent Document 1).

### PRIOR ART

Patent Document 1: Japanese Patent Application Publication No. 2019-58625

### SUMMARY OF THE INVENTION

However, the technology of Patent Document 1 can analyze the emotions of a target person but cannot estimate their specific mental state.

The present invention has been made in view of such background and aims to provide a technology capable of estimating a user's mental state.

The main invention of the present invention to solve the above problem is an information processing system comprising: a detection unit that analyzes a video image capturing a user and detects a degree of an attribute of the user; a calculation unit that calculates a second variation degree of a first variation degree related to the degree of the attribute in the video image; and an estimation unit that estimates a mental state of the user based at least on the second variation degree.

Other problems disclosed in this application and their solutions will be clarified by the description of the embodiments and the drawings.

According to the present invention, it is possible to estimate a user's mental state.

### BRIEF DESCRIPTION OF DRAWINGS

FIG. 1 is a diagram showing an overall configuration example of an information processing system.

FIG. 2 is a diagram showing a hardware configuration example of a management server 2.

FIG. 3 is a diagram showing a software configuration example of the management server 2.

FIG. 4 is a diagram explaining the operation of the management server 2.

FIG. 5 is a diagram showing an example of SHAP analysis results.

### DETAILED DESCRIPTION OF THE INVENTION

### <System Overview>

The following describes an information processing system according to an embodiment of the present invention. The information processing system of this embodiment aims to estimate a user's mental state (particularly the degree of depression) from a video capturing the user.

In this embodiment, “mental state” refers not merely to temporary physiological states (such as drowsiness or fatigue) or cognitive function states (such as concentration or attention), but to more persistent emotional and psychological health states. Specifically, it includes emotional aspects such as degree of depression, anxiety level, and stress level, and is a concept related to severity evaluation of mood-related disorders such as depression and anxiety disorders in psychiatric terms. Mental state is a concept that captures emotional and mood fluctuations, clearly distinguished from cognitive states such as “concentration” and “attention” which result from the allocation of cognitive resources, or physiological states such as “drowsiness” and “fatigue” which mainly result from physical arousal levels.

In conventional technology, for example, in drowsiness detection systems or concentration monitoring systems, attributes such as blinking frequency or degree of eye opening were used to detect temporary states. In contrast, the “mental state” estimated in this embodiment targets emotional and psychological states that persist for several days to several weeks, rather than temporary cognitive states.

Additionally, the evaluation of such mental states has traditionally relied mainly on self-reports or physician evaluations using questionnaires such as QIDS (Quick Inventory of Depressive Symptomatology), PHQ-9 (Patient Health Questionnaire-9), MADRS (Montgomery-Asberg Depression Rating Scale), etc. The information processing system of this embodiment provides an objective evaluation method that can replace or complement these conventional evaluation methods. In particular, by using the second variation degree of the degree of an attribute (such as the standard deviation of a standard deviation), it quantitatively evaluates the stability/instability of emotional expression, thereby estimating mood-related mental states. This approach enables the evaluation of deeper emotional health states rather than merely detecting temporary cognitive states.

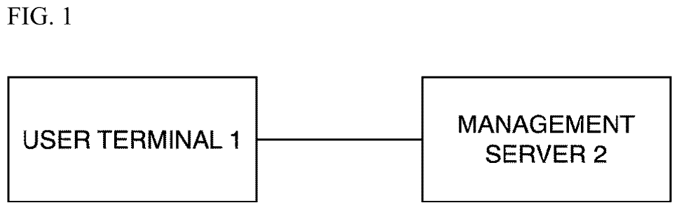

FIG. 1 is a diagram showing an overall configuration example of the information processing system. The information processing system of this embodiment includes a management server 2. The management server 2 is connected to a user terminal 1 via a communication network to enable communication. The communication network is, for example, the Internet, built using public telephone networks, mobile phone networks, wireless communication channels, Ethernet (registered trademark), etc.

The user terminal 1 is a computer operated by a user. The user terminal 1 can be, for example, a smartphone, a tablet computer, a personal computer, etc.

The management server 2 is a computer that estimates the user's mental state. The management server 2 may be a general-purpose computer such as a workstation or personal computer, or may be logically implemented through cloud computing.

### <Management Server>

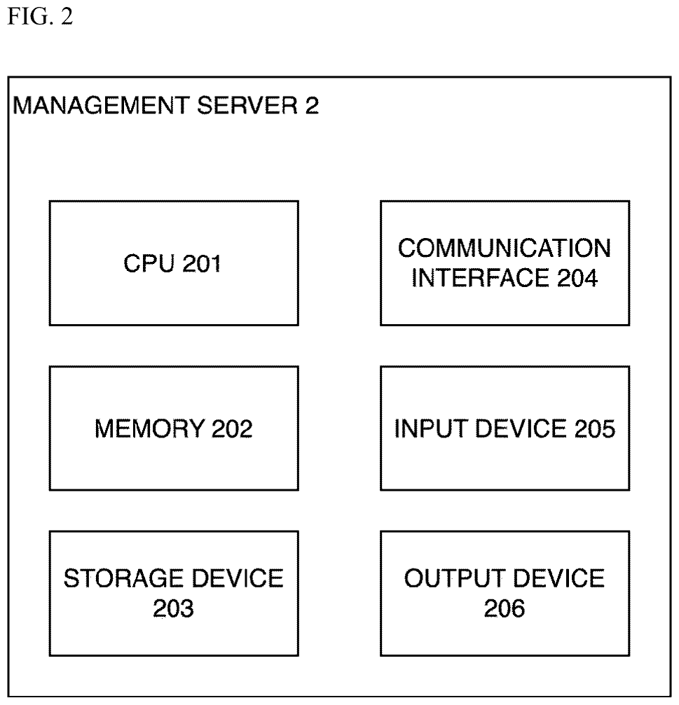

FIG. 2 is a diagram showing a hardware configuration example of the management server 2. Note that the configuration shown is an example and may have other configurations. The management server 2 includes a CPU 201, memory 202, storage device 203, communication interface 204, input device 205, and output device 206. The storage device 203 stores various data and programs, and is, for example, a hard disk drive, solid-state drive, flash memory, etc. The communication interface 204 is an interface for connecting to a communication network, for example, an adapter for connecting to Ethernet (registered trademark), a modem for connecting to a public telephone network, a wireless communication device for wireless communication, or a USB (Universal Serial Bus) connector or RS232C connector for serial communication. The input device 205 inputs data, for example, a keyboard, mouse, touch panel, button, microphone, etc. The output device 206 outputs data, for example, a display, printer, speaker, etc. Note that each functional part of the management server 2 is implemented by the CPU 201 reading and executing programs stored in the storage device 203 into the memory 202, and each storage part of the management server 2 is implemented as part of the storage area provided by the memory 202 and the storage device 203.

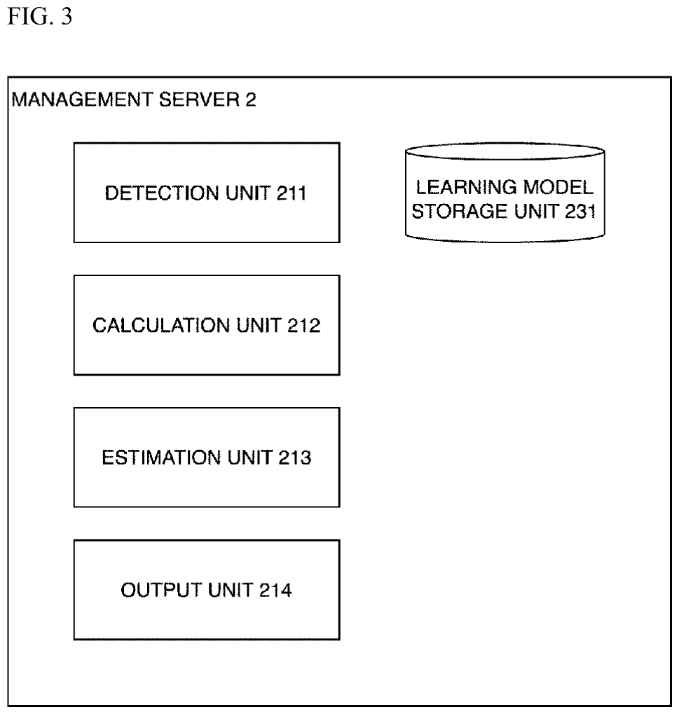

FIG. 3 is a diagram showing a software configuration example of the management server 2. The management server 2 includes a learning model storage unit 231, a detection unit 211, a calculation unit 212, an estimation unit 213, and an output unit 214.

### <Storage Unit>

The learning model storage unit 231 stores a first learning model for detecting the degree of a user's attribute (hereinafter referred to as attribute degree) from a video image, and a second learning model for estimating the user's mental state based on the detected attribute degree.

In this embodiment, the first and second learning models are created through machine learning. Machine learning can be broadly divided into supervised learning and unsupervised learning. Supervised learning is a method of training a model using input data and corresponding output data (teaching data), adjusting the model's parameters based on the teaching data to learn the mapping from input data to output data. In contrast, unsupervised learning is a method of learning the structure or patterns of input data without teaching data, learning the density distribution or feature representation of input data. In this embodiment, for the first and second learning models, supervised learning methods such as neural networks, support vector machines, decision trees, random forests, etc. can be used. Alternatively, unsupervised learning methods such as self-organizing maps, k-means method, etc. can also be used. These machine learning algorithms process input data with weighting or transformation to optimize parameters to minimize the error with output data. This allows learning the mapping from input data to output data.

The first learning model can be created through machine learning using features extracted from video images and attribute degrees as training data. Features input to the first learning model include, for example, images of facial regions extracted from each frame of the video, the position and size of organs such as eyes, nose, and mouth extracted from the facial region, and temporal changes in these organs. On the other hand, the attribute degrees output by the first learning model include, for example, the number of blinks, degree of eye opening, degree of mouth opening, eyebrow position, face orientation, and temporal changes in these. Specifically, the first learning model can output attribute degrees such as the number of blinks or degree of eye opening using features such as facial region images or organ positions and sizes as input. Attributes may include the number of blinks, eye offset (angle of the eye relative to the camera), gaze estimated from eye offset, facial expressions, etc. Facial expressions may include anger, disgust, fear, happiness, sadness, surprise, neutral, negative/positive, etc., and can infer the average or median value of their appearance frequency or direction in a predetermined period (such as 1 second), or the degree of emotions (facial expressions) such as anger.

The second learning model is a learning model that estimates the mental state (degree of depression) when given at least one of the degree of an attribute (its value), a statistical value related to the degree of the attribute (such as standard deviation), and a statistical value of that statistical value (such as the standard deviation of a standard deviation). In this embodiment, the features given to the second learning model include at least a statistical value of a statistical value (such as the standard deviation of a standard deviation). The second learning model can be created through machine learning using at least one of the degree of an attribute, a statistical value related to the degree of the attribute, and a statistical value of that statistical value, along with mental states judged by experts, as training data.

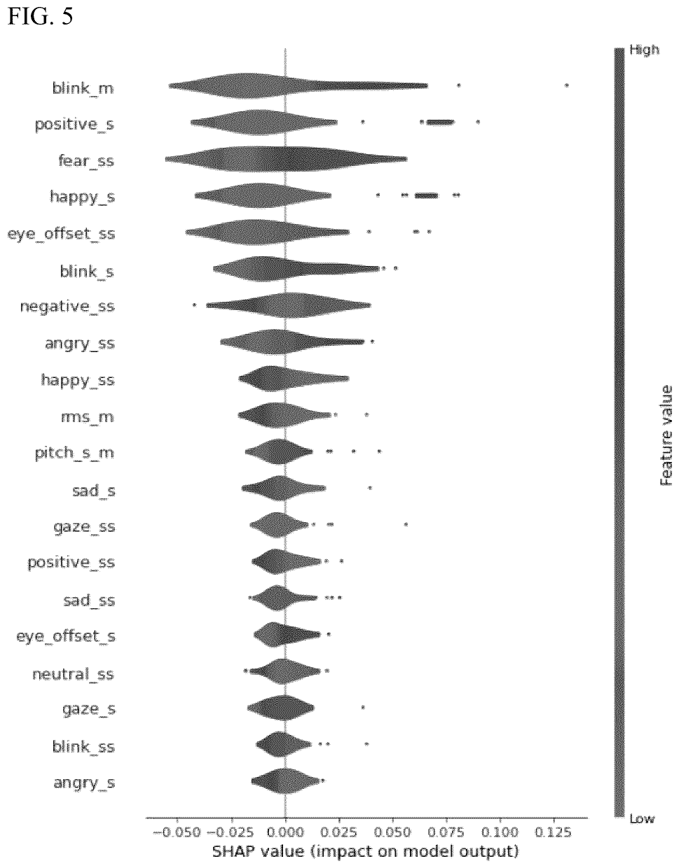

Furthermore, the inventors of this application conducted SHAP (SHapley Additive explanations) analysis to visualize the contribution of features input to the second learning model. FIG. 5 is a diagram showing an example of SHAP analysis results. As shown in FIG. 5, features representing the standard deviation of a standard deviation (hereinafter referred to as second-order variation degree) with the suffix “ss”—for example, blink_ss, fear_ss, positive_ss—showed higher SHAP value distributions than other mean values or first-order variation degrees (standard deviations) on model output, confirming that they are extremely important in estimating the user's degree of depression.

For example, it has been shown that users with depressive tendencies exhibit notable instability in fluctuations, particularly in certain emotional expressions (especially fear and sadness). Specifically, it can be said that while the fluctuation of emotional expression (standard deviation) is relatively stable in normal states, when the mental state deteriorates, the fluctuation itself becomes unstable (the standard deviation of the standard deviation increases).

The second-order variation degree is obtained by calculating the standard deviation of the degree of an attribute for each predetermined time window (e.g., 30 seconds), and then computing the standard deviation again for these standard deviation values per time window. This allows quantification of long-term instability that captures the “fluctuation” of attribute variation itself, distinct from instantaneous emotional reactions, thereby significantly enhancing the detection sensitivity of the degree of depression (or excitement).

### <Functional Units>

The detection unit 211 analyzes a video image to detect the degree of a user's attribute. The detection unit 211 can detect the degree of an attribute for each interval of predetermined length. The detection unit 211 can estimate the degree of an attribute by inputting the video image into the first learning model.

The calculation unit 212 calculates a second variation degree (second-order variation degree) of a first variation degree related to the degree of the attribute in the video image. Specifically, the calculation unit 212 calculates the first variation degree, which is the variation degree of the degree of the attribute (for example, the standard deviation of the degree of the attribute), and further calculates the second variation degree, which is the variation degree of the first variation degree (for example, the standard deviation of the standard deviation of the degree of the attribute). In this embodiment, the variation degree is assumed to be standard deviation, but it may be variance. For example, the calculation unit 212 can calculate the first variation degree, which is the standard deviation of the degree of anger, and the second variation degree, which is the standard deviation of that standard deviation.

The estimation unit 213 estimates the user's mental state based at least on the second variation degree. The estimation unit 213 can estimate the mental state based on the second variation degree and at least one of the first variation degree or the degree of the attribute. The estimation unit 213 can estimate the mental state by inputting at least the second variation degree into the second learning model.

The output unit 214 outputs the estimated mental state.

### <Operation>

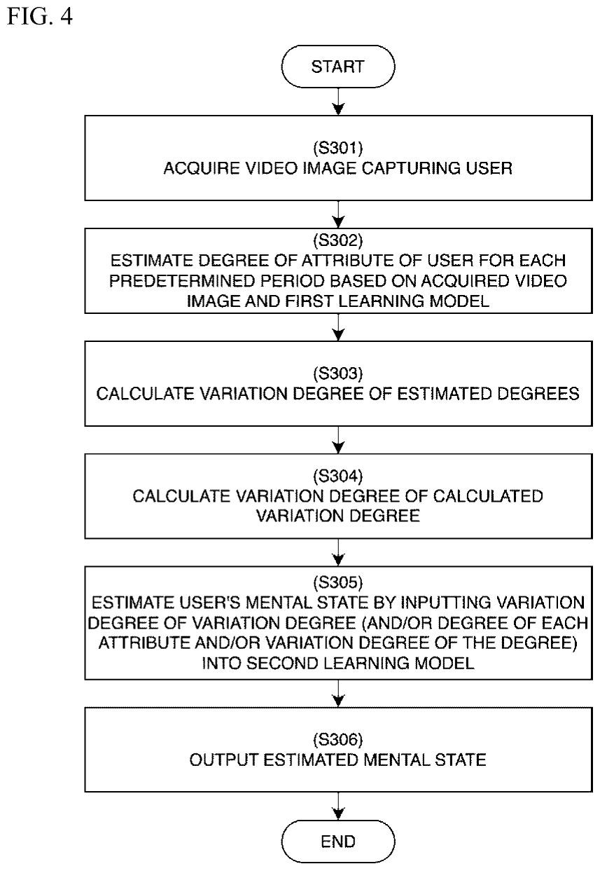

FIG. 4 is a diagram explaining the operation of the management server 2.

The management server 2 acquires a video image capturing the user (S301), estimates the degree of each attribute of the user for each predetermined period (for example, 1 second, etc.) based on the acquired video image and the first learning model (S302), and calculates the variation degree of the estimated degrees (S303). Here, standard deviation or variance can be used as the variation degree. For example, when calculating the standard deviation of the degree of an attribute, the calculation unit 212 calculates the standard deviation from the estimated degree of the attribute. On the other hand, when calculating the variance of the degree of an attribute, the calculation unit 212 calculates the variance from the estimated degree of the attribute. Next, the management server 2 calculates the variation degree of the calculated variation degree (S304). For example, when calculating the standard deviation of the standard deviation of the degree of an attribute, the calculation unit 212 calculates the standard deviation from the standard deviation of the degree of the attribute. On the other hand, when calculating the variance of the variance of the degree of an attribute, the calculation unit 212 calculates the variance from the variance of the degree of the attribute. Then, the management server 2 estimates the user's mental state by inputting at least the variation degree of the variation degree (and/or the degree of each attribute and/or the variation degree of the degree) into the second learning model (S305), and outputs the estimated mental state (S306).

As described above, according to the information processing system of this embodiment, it is possible to estimate a user's mental state from a video image capturing the user, allowing simple estimation of the mental state without using tests such as QIDS. Furthermore, according to the information processing system of this embodiment, estimation can be performed using the standard deviation of the standard deviation of the degree of an attribute as a feature. For example, users with deteriorating mental states may exhibit attitudes such as overreacting to certain topics while showing no interest in others, and by evaluating the standard deviation of the standard deviation (variation degree of the variation degree), it becomes possible to evaluate how much the variation degree of facial expressions, etc. fluctuates over time, which is expected to improve the accuracy of mental state estimation.

Additionally, according to the information processing system of this embodiment, by adopting not only the first variation degree but also the second-order variation degree of the degree of a user's attribute as a feature, it is possible to accurately capture the extent to which emotional expressions such as facial expressions, gaze, and blinking are “unstable” over time. As a result, even mild to moderate depression, which is often overlooked by conventional mean-centered methods, can be estimated with high accuracy, making it possible to improve the reliability of mental health screening.

The above embodiment has been described to facilitate understanding of the present invention and is not intended to limit the interpretation of the present invention. The present invention may be changed or improved without departing from its spirit, and the present invention includes its equivalents.

For example, the processing by each functional unit of the management server 2 described above may be executed by any functional unit. Also, different functional units that execute part of the processing of each functional unit may be added. Also, the functional units of the management server 2 may be distributed across multiple computers.

Also, the information stored in each storage unit of the management server may be stored in any storage unit. That is, information stored in multiple storage units described above may be stored by a single storage unit, or part of the information stored in one storage unit described above may be stored by another storage unit.

### Modification Example 1

In the above embodiment, the degree of a user's attribute was detected from a video image, but this is not limited to this. The degree of a user's attribute may be detected from audio in addition to the video image. For example, a user's voice can be acquired using an audio input device such as a microphone, and from the acquired voice, attributes such as the loudness of the user's voice, voice intonation, speaking speed, etc. can be detected. For the degree of voice loudness, for example, voice amplitude, sound pressure, volume, etc. can be used. For the degree of voice intonation, for example, changes in the fundamental frequency (pitch) of the voice can be used. For the degree of speaking speed, for example, the number of syllables or words per unit time can be used.

For the degree of an attribute based on voice detected in this way, as in the above embodiment, the first variation degree (such as standard deviation or variance) and the second variation degree (such as standard deviation of standard deviation or variance of variance) can be calculated, and these values can be used to estimate the mental state. For example, the estimation unit 213 can estimate the user's mental state using the degree of an attribute detected from audio in addition to the degree of an attribute detected from the video image, and their variation degrees. This makes it possible to improve the accuracy of mental state estimation using information that cannot be obtained from just the video image.

### Modification Example 2

In the above embodiment, a single attribute degree (for example, the degree of smiling, the degree of eye opening, etc.) was used as the degree of an attribute, but a value combining multiple attributes may be used as the degree of an attribute. For example, the detection unit 211 can detect the degree of smiling and the degree of eye opening from a video image, and calculate a value combining these values as the degree of an attribute. The combination of the degree of smiling and the degree of eye opening can be, for example, a weighted sum of the degree of smiling and the degree of eye opening, or a product of the degree of smiling and the degree of eye opening.

For the degree combining multiple attributes calculated in this way, as in the above embodiment, the first variation degree (such as standard deviation or variance) and the second variation degree (such as standard deviation of standard deviation or variance of variance) can be calculated, and these values can be used to estimate the mental state. For example, the estimation unit 213 can estimate the user's mental state using the degree combining the degree of smiling and the degree of eye opening, and its variation degree.

Also, a value combining three or more attributes may be used as the degree of an attribute. For example, a value combining the degree of smiling, the degree of eye opening, and the loudness of the voice can be used as the degree of an attribute. This makes it possible to improve the accuracy of mental state estimation using complex information that cannot be obtained from a single attribute.

### Modification Example 3

In the above embodiment, the management server 2 acquired a video image from the user terminal and detected the degree of an attribute from that video image, but this is not limited to this. The user terminal 1 may detect the degree of an attribute from a video image and send the detected degree of an attribute to the management server 2.

Specifically, the user terminal 1, using an imaging device such as a camera, captures the user and, as in the above embodiment, detects the degree of the user's attribute from the captured video image. Then, the user terminal 1 sends the detected degree of the attribute to the management server 2. The degree of the attribute may be detected at predetermined time intervals (for example, every 1 second), and the degree of the attribute detected at each time interval may be sent to the management server 2.

The management server 2, based on the degree of the attribute received from the user terminal 1, calculates, as in the above embodiment, the first variation degree of the degree of the attribute (such as standard deviation or variance) and the second variation degree of the first variation degree (such as standard deviation of standard deviation or variance of variance), and estimates the user's mental state using the calculated variation degrees.

Also, it is possible to have the user terminal 1 include all the functional units and storage units of the management server 2 without providing a management server 2, allowing the user terminal 1 to detect the degree of the attribute and also detect the mental state. In this case, the management server 2 may have functions to manage the learning models and input data to the learning models, and may delegate the function of sending input data to the management server 2 to generate responses.

### Modification Example 4

In the above embodiment, the current mental state of the user was estimated based on the current degree of the user's attribute and its variation degree, but this is not limited to this. The future mental state of the user may be estimated based on the current and past degrees of the attribute and their variation degrees.

Specifically, the management server 2 acquires the degree of the attribute and its variation degree for a predetermined period up to the present (for example, the most recent week). Then, the management server 2, based on the acquired degree of the attribute and its variation degree, estimates not only the current mental state but also the future mental state.

As a method for estimating the future mental state, for example, a method can be considered where the future degree of the attribute and its variation degree are predicted from the current and past degrees of the attribute and their variation degrees, and the future mental state is estimated based on the predicted future degree of the attribute and its variation degree. For the prediction of the degree of the attribute and its variation degree, time series data analysis methods (such as ARIMA models, RNNs, etc.) can be used.

Also, the future mental state may be directly predicted from the transition of current and past mental states. For example, the estimation unit 213 can estimate the transition of mental states over a predetermined period up to the present from the degree of the attribute and its variation degree over a predetermined period up to the present, and predict the future mental state based on the estimated transition of mental states. Time series data analysis methods can also be used when predicting the future mental state from the transition of mental states.

### Modification Example 5

In the above embodiment, the degree of depression was estimated as the mental state, but this is not limited to this. Other indicators separately identifiable from the mental state, such as the degree of stress or concentration, may also be estimated.

The degree of stress can be estimated based on the degree of the user's attributes such as the number or frequency of blinks, the degree of eye opening, the degree of mouth opening, face orientation, the loudness or intonation of the voice, speaking speed, etc., and their variation degrees. Generally, users with high stress tend to have a high number or frequency of blinks, widely open eyes or mouth, unstable face orientation, unstable voice loudness or intonation, fast speaking speed, etc., so the degree of stress can be estimated from the degrees of these attributes and their variation degrees.

Concentration can be estimated based on the degree of the user's attributes such as the amount and speed of gaze movement, the degree of gaze fixation, the number or frequency of blinks, etc., and their variation degrees. Generally, users with high concentration tend to have a small amount and speed of gaze movement, long periods of fixed gaze on one point, a low number or frequency of blinks, etc., so concentration can be estimated from the degrees of these attributes and their variation degrees.

The estimation unit 213 can estimate the user's degree of stress or concentration using a learning model that has learned the relationship between the degree of the above attributes and their variation degrees, and the degree of stress or concentration.

### Disclosure Items

This disclosure also includes the following configurations.

### Item 1

An information processing system comprising:
a detection unit that analyzes a video image capturing a user and detects a degree of an attribute of the user;
a calculation unit that calculates a second variation degree of a first variation degree related to the degree of the attribute in the video image; and
an estimation unit that estimates a mental state of the user based at least on the second variation degree.

### Item 2

The information processing system according to item 1, wherein the detection unit detects the degree of the attribute for each interval of predetermined length.

### Item 3

The information processing system according to item 1, wherein the first variation degree and the second variation degree are represented by standard deviation.

### Item 4

The information processing system according to item 1, wherein the estimation unit estimates the mental state based on the second variation degree and at least one of the first variation degree or the degree of the attribute.

### Item 5

The information processing system according to item 1, wherein the estimation unit estimates the mental state by inputting at least the second variation degree into a learning model created by machine learning that uses at least the second variation degree and the mental state as training data.

### Item 6

A method executed by a computer for information processing comprising:
analyzing a video image capturing a user to detect a degree of an attribute of the user;
calculating a second variation degree of a first variation degree related to the degree of the attribute in the video image; and
estimating a mental state of the user based at least on the second variation degree.

### REFERENCE SIGNS LIST

1 User terminal
2 Management server

1. An information processing system comprising:
a detection unit that analyzes a video image capturing a user and detects a degree of an attribute of the user;
a calculation unit that calculates a second variation degree of a first variation degree related to the degree of the attribute in the video image; and
an estimation unit that estimates a mental state of the user based at least on the second variation degree.

2. The information processing system according to claim 1, wherein the detection unit detects the degree of the attribute for each interval of predetermined length.

3. The information processing system according to claim 1, wherein the first variation degree and the second variation degree are represented by standard deviation.

4. The information processing system according to claim 1, wherein the estimation unit estimates the mental state based on the second variation degree and at least one of the first variation degree or the degree of the attribute.

5. The information processing system according to claim 1, wherein the estimation unit estimates the mental state by inputting at least the second variation degree into a learning model created by machine learning that uses at least the second variation degree and the mental state as training data.

6. A method executed by a computer for information processing comprising:
analyzing a video image capturing a user to detect a degree of an attribute of the user;
calculating a second variation degree of a first variation degree related to the degree of the attribute in the video image; and
estimating a mental state of the user based at least on the second variation degree.
